(19) United States
(12) Patent Application Publication
(10) Pub. No.: US 2003/0186954 A1
(43) Pub. Date: Oct. 2, 2003

(54) Estriol to treat and prevent cardiovascular disease

(76) Inventor: R. Kent Hermsmeyer, Portland, OR (US)

Correspondence Address: HOWARD EISENBERG, 1600 ODS TOWER, 601 S.W. SECOND AVENUE, PORTLAND, OR, 97204-3157, US

(21) Appl. No.: 10/108,119

(22) Filed: Mar. 26, 2002

(51) Int. Cl. A61K 31/56

(52) U.S. Cl. 514/182

(57) ABSTRACT

A method for preventing myocardial ischemia by preventing or treating coronary arterial vasoconstrictor hyperreactivity in which an amount of estriol that is effective to prevent or eliminate the hyperreactivity is administered to a subject in need thereof, thereby preventing or treating coronary vasospasm or ischemia. Preferably, the estriol is administered topically.

### FIELD OF THE INVENTION

The invention relates to the use of hormones to treat or prevent cardiovascular disease, including coronary artery disease such as coronary artery reactivity. Particularly, the invention relates to the use of hormones of the estrogen family to treat and prevent such cardiovascular disease.

### BACKGROUND OF THE INVENTION

Cardiovascular disease, including coronary heart disease, stroke and other vascular diseases, is the leading cause of death of men and women in economically-developed countries. The most common and lethal form of cardiovascular disease is ischemic heart disease. It has generally been regarded that ischemic heart disease is caused, primarily, by atherosclerosis of the coronary arteries. This is a condition where plaques form in the inner lining of the arteries, causing narrowing of the channel and thereby impairing blood flow to the heart.

An increased risk for ischemic heart disease is observed in women after menopause or ovariectomy and presents a major medical challenge. It is the leading cause of death in post-menopausal women, which implicates the importance of the loss of ovarian steroid hormones.

Since it is understood that the formation of plaques is reduced, or even reversed by a high ratio of high density lipoproteins (HDL) to low density lipoproteins (LDL), it has been a strategy to inhibit ischemic heart disease by attempting to increase this ratio in the blood stream. Estradiol, an ovarian estrogenic steroid hormone, has been observed to increase the HDL/LDL ratio, and studies so far suggest that estrogen replacement therapy for post-menopausal women decreases the incidence of coronary artery disease, myocardial infarction and related cardiovascular events by up to 50%.

The mechanism of estradiol's cardioprotective effects is not completely established, although a favorable impact on the circulating lipid and lipoprotein profiles is postulated as central to estrogen's cardioprotective effects. This explanation, however, is acknowledged to account for only a minority of estradiol's potential cardioprotective effects.

A vasospasm is an abnormally strong and persistent contraction of the muscles of the coronary arteries which leads to transmural myocardial ischemia and can result in sudden cardiac death. The role of coronary vasospasm in cardiovascular disease is still controversial, and approaches to treatments for cardiovascular disease have not focused upon methods for reducing coronary vasospasm. Instead, it is generally believed that coronary vasospasm is caused by local injury to vessels, such as results from atherosclerosis and other structural injury, and that long-term treatment of cardiovascular disease requires prevention of atherosclerotic plaques, not treatments to prevent vasospasm.

Estradiol has been shown to control coronary vasospasms. However, one important issue is the danger of using unopposed estradiol, which is associated with an increased risk of developing endometrial bleeding, endometrial cancer, breast cancer, and liver disorders.

Hermsmeyer, U.S. Pat. No. 6,056,972, incorporated herein by reference and referred to hereafter as the “Hermsmeyer patent”, discloses that low levels of progesterone, either alone or in combination with estradiol, can inhibit coronary artery reactivity by a direct effect on coronary arteries and, therefore, can be used to inhibit certain adverse cardiovascular events and disorders. Progesterone, administered in conjunction with estradiol, dramatically reduces the incidence of the adverse effects of unopposed estrogen. Hermsmeyer discloses that coronary artery vasospasm can be prevented by administering to a subject progesterone in an amount to achieve blood levels of progesterone of between 0.1 nanograms/ml and less than 4 nanograms/ml for at least 4 hours per day, and wherein said amount results in peak levels of progesterone of less than 6 nanograms/ml. Hermsmeyer further discloses that progesterone may be used to treat an existing vasospasm.

Hermsmeyer discloses that exaggerated vasoconstrictions of long (greater than 5 minute) duration are reliably initiated by the synergistic combination of two major platelet release products, serotonin and thromboxane A₂ at concentrations found in platelets. Hermsmeyer discloses that administration of these two compounds to menopausal monkeys produces intense, focal constrictions that mimic arterial vasoconstrictions in humans. These induced vasoconstrictions are prevented or terminated by the administration of estradiol and/or progesterone.

Estriol (E₃) is an estrogenic steroid hormone that differs significantly from estradiol. Unlike estradiol, estriol is not associated with increased risk of cancer and other side effects. Also, unlike estradiol, estriol is metabolized by hepatic metabolism and excreted in the urine. Because estriol's properties differ from those of estrogen, little is known about therapeutic benefits of estriol, especially regarding the cardiovascular system.

A significant need exists for an estrogen that can be administered on a routine, long-term basis to reduce the risk of coronary artery vasospasm, especially in post-menopausal women, without increasing the subject's risk of the effects of unopposed estradiol, including breast and endometrial cancer.

### SUMMARY OF THE INVENTION

It has been surprisingly discovered that estriol blocks the formation of coronary vasospasm in individuals lacking a significant source of endogenous estrogen. According to one embodiment of the method of the invention, coronary arterial vasospasms are prevented by administering to a patient in need thereof an effective amount of estriol, thereby inhibiting the formation of coronary arterial vasospasms and preventing myocardial ischemia.

In another embodiment, the invention is a method for treating a patient experiencing a coronary arterial vasospasm by administering an amount of estriol effective to terminate the vasospasm.

In another embodiment, the invention is a kit for administering estriol to a patient. The kit includes a package which houses a container, a topical preparation containing estriol in the container, and instructions. The instructions are for dispensing an amount of the preparation which amount, when applied topically to the skin, provides an amount of estriol effective to prevent or terminate coronary arterial vasospasms. An optional component of the kit is a dispenser for dispensing a metered amount of the topical preparation.

In another embodiment, the invention is a kit for administering estriol to a patient by using a transdermal patch. The patch includes a housing, a reservoir in the housing, a membrane attached to the housing, adjacent the reservoir, for placement against the epidermis of a human subject, and an adhesive attached to the housing for holding the membrane to the epidermis of the subject. The kit preferably contains instructions for using the patch to treat a coronary condition. Estriol in a carrier is contained in the reservoir, and the patch is constructed and arranged to deliver an amount of estriol to the epidermis of the subject to achieve blood levels of estriol that are effective to prevent or treat coronary arterial vasospasms.

In any of the embodiments of the invention, the estriol may be applied together, either in the same or separate administration, with other hormone replacement therapy, such as another estrogen such as estradiol, testosterone or derivatives other than dihydrotestosterone (DHT), or a progestin such as progesterone.

These and other aspects of the invention are described in greater detail below.

### BRIEF DESCRIPTION OF THE FIGURES

FIG. 1 is an angiogram depicting the absence of vasospasm in a coronary artery.

FIG. 2 is an angiogram showing a vasospasm induced in a primate animal model of human coronary arterial vasospasm disease by the administration of a combination of serotonin and U46619.

### DETAILED DESCRIPTION OF THE INVENTION

The invention relates to coronary artery reactivity and, in one aspect, involves the use of estriol to prevent or treat coronary artery vasospasm, thereby preventing cardiovascular disorders.

A cardiovascular disorder as used herein means myocardial infarction, ischemic heart disease, heart failure, stroke, angina pectoris, and peripheral arterial vascular disease. Coronary reactivity, as used herein, is a measure of the amplitude and duration of a response of an artery to an applied vasoconstrictor stimulus. According to the present invention, methods are provided for reducing the reactivity of coronary arteries to a vasoconstrictive substance, thereby preventing or treating a coronary vasospasm. The methods of the invention thus are adapted to prevent or relieve coronary vasospasm, a focal constriction that is hypothesized to result from local vascular hyperreactivity to vasoconstrictive substances.

Coronary artery reactivity can be measured indirectly or directly. Indirect measures include a cell's response, such as a vascular smooth muscle cell's response to serotonin (5 HT) and a thromboxane A₂ mimetic, such as U46619, applied in vitro as described herein and in the Hermsmeyer patent. Direct measures include in vivo animal vascular responses to conditions for inducing a coronary vasospasm, e.g. in vivo mechanical injury or in vivo treatment of a hyperreactive animal with a combination of serotonin and U46619, also described herein and in the Hermsmeyer patent.

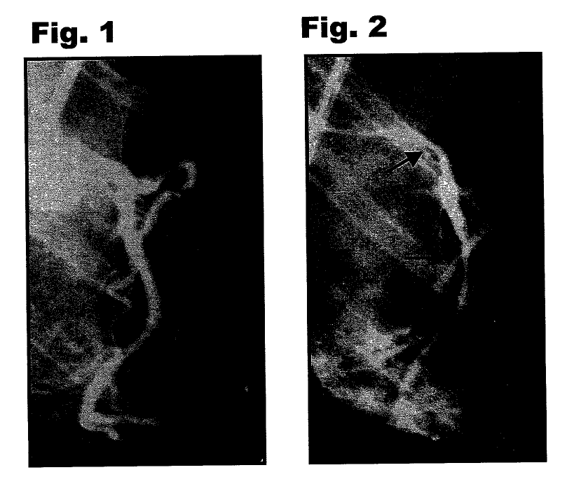

A vasospasm is an abnormally strong and persistent contraction of the muscles of the coronary arteries which leads to transmural myocardial ischemia and often results in sudden cardiac death. The vasospasm causes the coronary artery to assume a characteristic “hourglass” shape of prolonged constriction. See FIG. 2 and compare with a coronary artery without a vasospasm in FIG. 1. The term “vasospasm” is often misused in the literature to refer to a vasoconstriction which, rather than being abnormal and life-threatening, is a normal, healthy contraction as a means of autoregulating blood flow. As used herein, a coronary vasospasm is defined as epicardial coronary arterial constriction to less than 33%, and preferably less than 25%, of control diameter in focal areas with adjacent downstream dilation, with the hourglass pattern thus formed persisting for >5 minutes.

It is noted that the concept of coronary artery reactivity has been used in the literature in a sense that is different from how it is used in this specification. Williams et al., J. American College of Cardiology, 24(7):1757-1761 (1994), uses this term to mean vasodilator capacity in response to acetylcholine, which causes dilation of normal coronary arteries but constriction or no effect in diseased arteries. According to this usage, decreases in reactivity are abnormal. Hyperreactivity, as used herein, refers to an exaggerated amplitude and/or duration of a response to a vasoactive substance. Therefore, hyperreactivity to a vasoconstrictive stimulus underlies coronary ischemia and vasospasm; decreased vasodilator response to stimuli, as found in atherosclerotic monkeys by Williams et al., is a separate and distinct mechanism by which coronary arteries fail to open, or to remain open, to provide for increases in coronary blood flow.

It has been found, surprisingly, that, in accordance with the invention, the effective amount of estriol that may be administered is very low and that amounts administered to produce a blood level about 100 pg/ml or even lower are effective to prevent or treat coronary arterial vasoconstrictor hyperreactivity, and thus coronary ischemia or vasospasm. Levels higher than 100 pg/ml are likewise effective.

Although the present invention has arisen out of studies in female rhesus monkeys, it will be understood by a person of ordinary skill in the art, that the results of these studies are directly applicable to human beings, especially to female humans. This is because female rhesus monkeys have been established as an excellent animal model for the physiological effects of female sex hormones on women. These experiments also provide a basis for estriol as a prophylactic treatment for myocardial ischemia in men.

Thus, the invention is useful in men and women. The invention is particularly useful in women who have abnormally low levels of estrogen and progesterone, due to natural circumstances, surgery, or disease. Typically such women are post-menopausal or ovariectomized and may suffer from hot flashes and night sweats. Such women can be otherwise healthy. In particular, such women may or may not be hypercholesterolemic. In this regard, the invention has provided the surprising finding that coronary hyperreactivity can be independent of atherosclerosis, and estriol can have its effect directly on an artery, not via lipid pathways.

Animal studies using female rhesus monkeys, described in the Hermsmeyer patent, have revealed that coronary vasospasm occurs, in the absence of injury, plaques or other vascular pathology, due to local regions of vascular muscle hyperreactivity. In particular, vasospasm occurs in rhesus monkeys in the absence of atherosclerosis, which demonstrates that pure reactivity is sufficient to account for practically stopping vital coronary blood flow. Coronary vasospasm leads to transmural myocardial ischemia and can result in sudden cardiac death. Moreover, it has been found that such vascular muscle hyperreactivity can be reproducibly elicited by the provocation, or challenge, with intracoronary injection of certain vaso-constricting agents. Thus, it has been found in these rhesus monkeys that are fed high fiber diets which prevent atherosclerosis, that life-threatening myocardial ischemia resulting from coronary hyperreactivity or vasospasm can be stimulated by drugs in the absence of atherosclerosis or other vascular pathology.

The invention is useful in subjects who are otherwise apparently healthy and in those who are not apparently healthy. Apparently healthy, as used herein, means individuals who have not previously had an acute adverse cardiovascular event such as a myocardial infarction (i.e. individuals who are not at an elevated risk of a second adverse cardiovascular disorder due to a primary cardiovascular event). Apparently healthy individuals also do not otherwise exhibit symptoms of disease. In other words, such individuals, as examined by a medical professional, would be characterized as healthy and free of symptoms of disease.

The invention likewise is useful in “non-hypercholesterolemic subjects” and in “hypercholesterolemic subjects”. Non-hypercholesterolemic subjects do not fit the current criteria established for a hypercholesterolemic subject. Hypercholesterolemic subjects are associated with increased incidence of a cardiovascular disorder. A hypercholesterolemic subject has an LDL level of greater than 190 mg/dl, or greater than 160 mg/dl and at least two risk factors selected from the group consisting of a family history of premature coronary heart disease, cigarette smoking (more than 10 per day), hypertension, low HDL (<35 mg/dl), diabetes mellitus, hyperinsulinemia, abnormal obesity, high lipoprotein (a) and personal history of cerebral vascular disease or occlusive peripheral vascular disease, or greater than 130 mg/dl if ischemic heart disease is present.

The invention thus is useful in connection with treating populations of patients never before treated with estrogen. Such patients can be free of symptoms calling for estrogen treatment.

The invention involves the administration of estriol to subjects at a level effective to prevent or treat coronary arterial vasospasm. Preferably, sufficient estriol is administered to provide a blood level of estriol of about 100 pg/ml, which is well below the 250 to 500 pg/ml level reported as the desired blood level of estriol in clinical studies related to symptoms other than those addressed by the present invention. If desired, blood levels higher or lower than 100 pg/ml may be used in accordance with the present invention. For example, it is conceived that blood levels as low as about 20 pg/ml are effective in accordance with the method of the invention. Preferably, a blood level of estriol of between about 20 pg/ml and about 1000 pg (1 ng)/ml or higher is obtained. More preferably, a blood level between 50 pg/ml and 500 pg/ml is obtained. Most preferably, the blood level of estriol obtained following administration according to the method of the invention is between 75 pg/ml and less than 400 pg/ml.

Estriol is believed to be rapidly cleared from the bloodstream and quickly conjugated and eliminated by the kidney, which excretes estriol as a glucuronide or sulfate. Therefore, surprisingly, the benefit of estriol is conceived to result from only one or two passes through the coronary arteries at the doses described above.

It is also conceived that the effective blood levels obtained by administration of estriol in accordance with the invention may be reduced if estriol is administered in combination with other estrogens or with a progestin, which estrogen or progestin is effective in reducing or treating coronary arterial hyperreactivity or vasospasm. Estriol does not interfere with the cardiovascular protective effects of other estrogens and progestins such as progesterone and, in fact, independently acts, either alone or in combination with estrogen and/or progesterone, most probably at both a genetic level and a direct level on vascular smooth muscle cells to reduce the reactivity of the coronary artery constrictor response amplitude and duration.

To achieve optimal prophylactic effects, estriol may be administered continuously to provide the desired blood levels. It has been discovered surprisingly, that estriol applied topically can achieve the desired levels of estriol in the blood stream for at least 4, 6, 12 and even 24 hours with only a single application. Other forms of estriol administration, such as oral or intravascular administration, are less preferred because they are inconvenient and because they do not provide continuous blood estriol levels.

The preferred formulation is a topical preparation. As used herein, “topical” means applied externally to the surface of the skin. Specifically excluded in the definition of “topical” are cavities such as the vaginal, rectal or oral cavity. Also excluded is the corneum. The estriol is preferably dissolved in a water base in a combination of propylene glycol, sorbitol, and cetyl and stearyl alcohols. The estriol may be dissolved in a non-polar oil. Vitamin E (tocopherol) is one such preferred non-polar oil that may be used for this purpose in the formulation. Aloe vera and other water-based substances, such as emolients, may be included as additives to achieve a pleasant skin cream.

The effectiveness of estriol in such a cream as a delivery system is believed to be due, at least in part, to the combination of direct transdermal absorption of estriol into the subject's bloodstream and the slow rate of transdermal absorption. As a consequence, estriol in the cream formulation applied to the skin is absorbed and released over many hours, and is released directly into the blood stream so that it is not first metabolized into an inactive form by the liver, as would happen to estriol administered by the oral route, and then absorbed by the digestive system.

Topical preparations, as known in the art, typically are non-solid, liquid, cream, gel, lotion, or ointment preparations. They may contain skin penetration enhancers. Skin penetration enhancers are agents that when co-applied with a drug to the skin enhance the ability of the drug to penetrate the skin and be delivered into the blood stream. Skin penetration agents are discussed in Remington's Pharmaceutical Sciences, Mack Publishing Co., 18th Edition, Easton, Pa., USA (1990), which is incorporated herein by reference.

Patch technologies also may be used as a delivery system for estriol. Transdermal patches typically include a housing, a reservoir in the housing and a membrane attached to the housing adjacent the reservoir for placement against the epidermis of the human subject. The patch has also included an adhesive attached to the housing for holding the membrane to the epidermis of the subject. Patches suitable for use in the present invention and capable of delivering estriol in the amounts according to the invention may be found in the following prior art patents: U.S. Pat. No. 3,731,683; U.S. Pat. No. 3,797,494; and U.S. Pat. No. 4,336,243; U.S. Pat. No. 4,628,052; U.S. Pat. No. 4,704,282; U.S. Pat. No. 4,788,062; U.S. Pat. No. 4,906,169; and U.S. Pat. No. 5,164,190, the disclosures of which are incorporated herein by reference.

The use of estriol, and especially in a topical formulation such as a cream, has several advantages over conventional estrogen therapy, including 1) freedom from carcinogenic actions on breast or uterus, 2) lack of dysfunctional bleeding, 3) avoidance of major metabolic challenges to the liver, 4) lack of adverse effects on blood lipids, 5) lack of adverse effects on venous thromboembolism, 6) improvement of urogenital climacteric symptoms, and 7) improved compliance by post-menopausal women and thus more effective hormone replacement therapy (HRT). Conventional delivery systems, that is oral estrogen administration, has been found to have a very low compliance rate, typically only about 15%, even counting short duration use. In fact, the average duration of use is only 3 months, rather than for many years, which contributes to the fact that cardiovascular disease is the number one cause of death in women over 50 years of age.

A variety of administration routes for estriol besides the topical route, of course, are acceptable. The methods of the invention generally speaking, may be practiced using any mode of administration that is medically-acceptable, meaning any mode that produces the desired levels of estriol without causing clinical unacceptable adverse effects. Such modes of administration include oral, rectal, vaginal, topical, sublingual, nasal, intradermal or other parenteral routes such as inhalation. Intravenous, intramuscular and oral routes are not particularly preferred or suitable for long-term therapy and prophylaxis.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a lipid carrier, a finely divided solid carrier, or both, and then, if necessary, shaping a product. As mentioned above, formulations suitable for various modes of administering can be found in Remington's Pharmaceutical Sciences.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. They include polymer base systems such as poly(lactide-glycolate), copolyoxalates, polycaprolactones, polyester amides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, as used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

The invention also includes various kits. Each of the kits preferably includes instructions for dispensing an amount of estriol effective to prevent or treat coronary arterial vasospasm, such as the amounts to achieve the blood levels as described above. Each of the kits also contains in it a preparation of estriol, either constructed and arranged to deliver the appropriate amounts of estriol or with a dispensing means permitting dispensing of appropriate amounts of estriol. Thus, the kit includes a package which preferably houses instructions as described above. The kit also includes a topical preparation of estriol contained in a container which may be a bottle. A container may be any device for containing the estriol, such as ajar, bottle, vial, tube, packet and the like. This kit also optionally includes a syringe which may be used to withdraw from the container the appropriate amount of the topical preparation of estriol for use according to the invention.

In one preferred embodiment, the optional metering device is a syringe. The metering device, however, may be any such device known in the art for dispensing a metered amount of the preparation, or the kit may lack a metering device. For example, the metering device may be a calibrated pump that is attached to the container (i.e., a bottle with a pump dispenser) which is capable of delivering a metered amount of estriol according to the invention. The dispensing device also may be simply a measuring cup or vial, or pressurized aerosol bottle. Any means for dispensing a predetermined amount of the estriol is useful according to the invention.

A topical cream, gel, lotion, or ointment containing the estriol also may be contained in individual packets, each packet containing an appropriate dose for topical application. The kit may include a plurality of such packets, such as 30, 60, 100 or more packets, each packet containing a predetermined amount of cream, such as between about 1.2 to 3.2 ml, preferably about 2 ml of cream to deliver about 1.0 to 3.0 ml when squeezed, which in turn contains on the order of about 1 to 10 mg of estriol, preferably 2 to 5 mg of estriol, and most preferably about 3 mg of estriol, assuming a 0.3% estriol concentration in the cream. The kits also may include transdermal patches, which have been described in detail above, as the means for both containing and the means for dispensing the estriol.

The invention is further disclosed in the following non-limiting examples.

### EXAMPLES

### Example 1

### Animals

Six adult ovariectomized female rhesus monkeys (Macaca mulatta) were employed in the studies described below. None of the monkeys had been exposed to high cholesterol diets and all were later verified to lack evidence of cardiovascular disease. Each of the monkeys had a normal physical examination, including blood chemistry analysis. The monkeys were entered into the study no less than 3 months after ovariectomy. Monkeys do not endogenously produce estriol, and therefore all blood or urine levels found must have resulted from exogenous administration.

### Example 2

### Catheterization

Angiography at the Dotter Institute, after overnight fasting, began with preanesthesia with ketamine (10 mg/kg body weight intramuscular), intubation with an endotracheal tube and a light surgical plane of anesthesia with isoflurane (induced at 1.5% to 3% maintained at 0.75% to 1.25%), vaporized with 33% nitrous oxide and 67% oxygen, or with 100% oxygen. Bilateral femoral intraarterial 3F catheters allowed for coronary catheterization and continuous recording of systemic arterial blood pressure and heart rate, which were maintained close to the initial anesthetic value for each monkey. Intravenous heparin (1,000 U) was injected, and up to 120 ml of pyruvated Ringer's solution and 0 to 50 ml of dextran solution were used (as needed) to maintain diastolic blood pressure ≧60 mm Hg. A heating pad assisted in maintenance of a body temperature decrease ≦2° C. of preanesthesia body temperature (monitored by rectal thermometer). An electrocardiogram (ECG) recorded on a Gould eight-channel recorder, cutaneous arterial oxygen saturation, respiratory rate and end-tidal CO₂ were also monitored.

Entire experiments were recorded on videotape (with episodic fluoroscopic imaging and continuous voice annotation) to permit computerized quantitative coronary angiography. For reasons explained in the Hermsmeyer patent, the agent for stimulating the production of coronary arterial vasospasm in susceptible monkeys was a combination of serotonin and U46619.

Placement of the 3F catheter (except as noted later) was adjusted to provide sufficient filling with radio-opaque contrast medium, limited reflux and isolation of a branch of the coronary arterial tree. Usually the left anterior descending coronary artery (LAD) was chosen, but the left circumflex or right coronary artery may alternatively be used. After adjustment of the camera angle to optimally image the coronary vascular tree, warm (35 to 38° C.) 1- to 2-ml boluses of Hexabrix (Mallinckrodt) radio-opaque contrast media were injected rapidly by hand to fill optimally. Fluoroscopic images were recorded on film using an OEC model 9800 digital x-ray system with fluoroscopic C-arm, digitally recorded, and evaluated in subsequent analysis. All procedures including a brief 0.2 ml injection of Hexabrix for optimal quantitative angiography were recorded on videotape for subsequent computer analysis.

Every injection of drugs was made by intra-coronary (IC) route, with a slow, constant flow of 1 ml over 30±1 second. The time between drug injections was typically 7 to 10 minutes, and no less than 4 minutes, with pressures and heart rates allowed to return to <15% change from baseline before the next injection. All concentrations were syringe concentrations (uncorrected for dilution by coronary blood flow) to exactly describe the procedure.

### Example 3

### Effect of Estriol to Prevent Vasospasm

A 0.3% estriol topical cream was formulated with the following components.

| Component | % Concentration w/w |
|---|---|
| estriot | 0.3 |
| cetyl alcohol, NF | 6 |
| stearyl alcohol, NF | 3 |
| sodium lauryl sulfate, NF | 0.2 |
| isopropyl palmitate, NF | 1.7 |
| propylene glycol, USP | 8 |
| sorbitol solution, USP | 12.9 |
| lactic acid, USP | 1.2 |
| benzyl alcohol, NF | 2.1 |
| purified water, USP | QS 100 |

The six rhesus monkeys of Example 1 were treated daily with this 0.3% estriol topical cream. The cream was rubbed into the skin of the monkeys on an area that had been shaven. This dosage of skin cream is expected to result in an absorption of about 0.3 mg estriol per ml, based on an assumption of 10% absorption of the 3 mg of estriol in each ml of cream. The estriol was purchased from Steraloids (Wilton, N.H.).

After 4 weeks of daily treatment with 0.2 mg/kg of the estriol topical cream, the monkeys were studied by coronary catheterization, and reactivity was assessed by injection of serotonin (100 μM) and U46619 (1 μM). Repeat slow intracoronary infusions of 1 ml over 30 seconds of the combination of serotonin and U46619 in this 100:1 molar ratio were used, adjusted for body weight (0.2 mg/kg) to produce coronary blood levels of 6.7 μM serotonin and 67 nM U46619. The levels of these two components were based on calculated coronary flow dilution. Venous blood samples were collected just prior to beginning the study and after 2 weeks and 4 weeks of treatment for measurements of progesterone and estriol. Urine samples were collected just prior to cardiac catheterization.

Angiography was carried out under isoflurane (0.75-1.25%) general anesthesia with 70% O₂ and 30% N₂O. After ketamine sedation (10 mg/kg) and endotracheal tube placement, both femoral arteries were cannulated for simultaneous measurement of systemic blood pressure and coronary catheterization. Intravenous anticoagulation with 1000 units of heparin and fluid replacement with 75-150 ml of pyruvated Ringer's solution, and if needed 10-50 ml of dextran, to reach a minimum control diastolic blood pressure of 60 mm Hg.

Coronary arteries (left anterior descending or left circumflex) of the monkeys were catheterized and agents injected as described above in Example 1.

Statistical analysis was carried out by Student's t-test (non-paired) or Chi-square, with the p<0.05 level accepted as significant.

Table 1 shows the major finding of this study, which is that estriol protected against vasospasm. None of the monkeys (6 of 6) that had been exposed to estriol suffered a vasospasm during the study. As disclosed in the Hermsmeyer patent, the combination of serotonin and U46619 predictably produces vasospasm in all unprotected animals to which it is administered. Therefore, the results prove decisively that estriol is effective in preventing coronary vasospasm.

TABLE 1
Drug-induced Coronary Vasospasm in ovariectomized animals
Incidence and Steroid Levels

| Parameter | Estriol | No Estriol* |
|---|---|---|
| Vasospasm criteria met | 0/6 | 6/6** |
| Estradiol (E₂) in plasma, | 21 +/− 2 pg/ml | <8 pg/ml |
| pg/ml | | |
| Progesterone in plasma, | 0.7 +/− 0.3 ng/ml | <0.3 ng/ml |
| pg/ml | | |
| E₃ absorbed, urine | ***2412 +/− 316 (n = 5) ng/ml | <1 n /ml |
| ng/ml (total) | | |

*Control animals are historical controls. Such controls are disclosed in the Hermsmeyer patent
**Indicates significant differences between Estriol and No Estriol at p 0.05 (N = 6 for each group).
***Different from historical controls at p < 0.05 with unpaired t-test

The data shows that protection against coronary vasospasm by administration of estriol was categorical, as none of the E₃ treated monkeys showed a vasospasm in a 12 step intracoronary serotonin+U46619 injection protocol. For all historical controls, each of which received only a placebo treatment, all 6 of these monkeys showed coronary vasospasms that met the criteria of <33% of control diameter for >5 minutes.

### Example 4

### Levels of Estriol in Human Volunteers Treated with Skin Cream

Four human volunteers applied one of three estriol skin cream formulations in a base of polyethylene glycol, sorbitol, and water. The formulations were as described above in Example 3, except that one formulation contained 0.1% estriol, a second formulation contained 0.5% estriol, and a third contained 1% estriol. Each volunteer applied 4 ml of one of the formulations daily for 2 days for a total applied dosage of 4 mg, 20 mg, and 40 mg of estriol, respectively.

Concentrations of estriol in saliva was measured 24 hours after the second application of the estriol cream formulations. The group that applied the 0.1% estriol averaged a salivary concentration of about 300 pg/ml. The 0.5% group averaged about 1500 pg/ml. The 1%group averaged about 4000 pg/ml. This data indicates that target circulating levels of estriol are obtained by topical administration.

In addition, salivary concentration of estriol was measured ten minutes following the application of 20 mg of estriol in skin cream. A concentration of 10 ng/ml was found, indicating rapid absorption.

Further modifications, uses, and applications of the invention described herein will be apparent to those skilled in the art. It is intended that such modifications be encompassed in the following claims.

1. A method for preventing myocardial ischemia comprising administering to a subject in need thereof an effective amount of estriol.

2. The method of claim 1 wherein the subject is additionally administered a progestin.

3. The method of claim 1 wherein the subject is additionally administered an estrogen other than estriol.

4. The method of claim 1 wherein the estriol is administered topically to the skin of the subject.

5. The method of claim 1 wherein the amount of estriol administered results in a blood level of estriol of at least 20 pg/ml.

6. The method of claim 5 wherein the blood level is between 20 and 1000 pg/ml.

7. The method of claim 5 wherein the blood level is between 50 and 500 pg/ml.

8. The method of claim 5 wherein the blood level is between 75 and 250 pg/ml.

9. The method of claim 1 wherein the subject is a woman.

10. The method of claim 9 wherein the woman has coronary hyperreactivity and/or is menopausal.

11. A method for treating coronary vasospasm comprising administering to a subject in need thereof an effective amount of estriol.

12. The method of claim 11 wherein the estriol is administered topically to the skin of the subject.

13 The method of claim 11 wherein the amount of estriol administered results in a blood level of estriol of at least 20 pg/ml.

14. The method of claim 13 wherein the blood level is between 20 and 11000 pg/ml.

15. The method of claim 14 wherein the blood level is between 50 and 500 pg/ml.

16. The method of claim 15 wherein the blood level is between 75 and 250 pg/ml.

17. The method of claim 111 wherein the subject is a woman.

18. The method of claim 17 wherein the woman is menopausal.

19. A method for reducing coronary artery reactivity comprising administering to a subject in need thereof an effective amount of estriol.

20. The method of claim 19 wherein the estriol is administered topically to the skin of the subject.

21. The method of claim 19 wherein the amount of estriol administered results in a blood level of estriol of at least 20 pg/ml.

22. The method of claim 21 wherein the blood level is between 20 and 1000 pg/ml.

23. The method of claim 22 wherein the blood level is between 50 and 500 pg/ml.

24. The method of claim 23 wherein the blood level is between 75 and 250 pg/ml.

25. The method of claim 24 wherein the subject is a woman.

26. The method of claim 25 wherein the woman is menopausal.

27. A kit for reducing or preventing coronary vasospasm comprising a container, a pharmaceutical preparation containing estriol in the container, and instructions for dispensing an amount of the preparation which amount, when administered to a subject, provides an amount of estriol effective to prevent or reduce coronary arterial hyperreactivity or vasospasms.

28. The kit of claim 27 which further comprises a dispenser for dispensing a metered amount of the pharmaceutical preparation.

29. The kit of claim 27 wherein the pharmaceutical preparation is a topical preparation and the instructions are for dispensing the preparation to the skin.

30. The kit of claim 29 wherein the pharmaceutical preparation is in a form selected from the group consisting of a cream, gel, lotion, or ointment.

31. The kit of claim 29 wherein the topical preparation is in the form of a transdermal patch.
